(19) United States
(12) Patent Application Publication
(10) Pub. No.: US 2026/0368893 A1
(43) Pub. Date: Nov. 19, 2026

(54) METHOD AND ELECTRONIC DEVICE FOR CORRECTING PRESSURE DIFFERENCE VALUE OF BLOOD VESSEL

(71) Applicant: Medipixel, Inc., Gangnam-gu (KR)

(72) Inventors: Min-Yeong KANG, Gangnam-gu (KR); Jong Hoe KU, Gangnam-gu (KR)

(21) Appl. No.: 19/725,772

(22) Filed: Jun. 30, 2026

Related U.S. Application Data
(63) Continuation of application No. PCT/KR25/10513, filed on Jul. 17, 2025.

(30) Foreign Application Priority Data
Jul. 19, 2024 (KR) 10-2024-0095523
Sep. 30, 2024 (KR) 10-2024-0132715

(51) Int. Cl. G06T 7/00; G06T 7/62

(52) U.S. Cl. CPC G06T 7/0012; G06T 7/62; G06T 2207/10116; G06T 2207/30101

(57) ABSTRACT

There is provided a method including obtaining at least one image of a blood vessel of a subject captured by using an imaging apparatus, and imaging information of the imaging apparatus regarding the at least one image, obtaining, based on the imaging information, height information in a gravity direction of the blood vessel included in the at least one image, calculating, based on the height information, a height difference value between a first point and a second point of the blood vessel, calculating, based on the height difference value, a pressure difference value due to gravity between the first point and the second point of the blood vessel, and correcting, based on the pressure difference value due to gravity, a pressure difference value between the first point and the second point of the blood vessel to obtain a corrected pressure difference value.

### CROSS-REFERENCE TO RELATED APPLICATIONS

This application is a continuation application of International Application No. PCT/KR2025/010513 designating the United States, filed on Jul. 17, 2025, in the Korean Intellectual Property Receiving Office and claiming priority to Korean Patent Application No. 10-2024-0095523, filed on Jul. 19, 2024, and Korean Patent Application No. 10-2024-0132715, filed on Sep. 30, 2024, in the Korean Intellectual Property Office (now the Ministry of Intellectual Property (MOIP)), the disclosures of which are incorporated by reference herein in their entireties.

### BACKGROUND

### 1. Field

The disclosure relates to a method and an electronic device for correcting a pressure difference value of a blood vessel.

### 2. Description of Related Art

A coronary artery is an artery surrounding the heart and may supply oxygen and nutrients to the myocardium, which is the muscle of the heart. That is, as oxygen and nutrients are continuously supplied from the coronary artery to the myocardium, the function of the heart may be performed. Accordingly, if an abnormality (e.g., a disease) occurs in the coronary artery and thus oxygen and nutrients are not properly supplied to the myocardium, a cardiovascular disease such as myocardial infarction may develop.

In order to rapidly and accurately diagnose lesions in blood vessels, such as stenosis of the coronary artery, blood flow feature values such as fractional flow reserve (FFR) may be used for diagnosis. For example, when a lesion develops in the coronary artery, blood flow passes through the lesion and pressure drops due to a loss of kinetic energy, i.e., perfusion pressure, resulting in a pressure difference between the distal and proximal portions of the lesion. This pressure difference may be measured using a pressure wire, which is FFR equipment.

However, in addition to the pressure loss due to the narrowing of the inner diameter of the lesion, a pressure (e.g., hydrostatic pressure) difference depending on the vertical position of the blood vessel may affect the measurement results. For example, during FFR measurement, the left anterior descending coronary artery (LAD), which is located at the front of the heart, has a pressure sensor positioned higher than the blood vessel inlet, so even if there is no pressure loss in blood flow at all, the measurement may indicate that pressure loss has occurred due to a hydrostatic pressure difference. In addition, in some segments of the left circumflex coronary artery (LCx) or the right coronary artery (RCA), the opposite phenomenon may occur.

That is, in the LAD, a problem of overestimating the FFR may occur, and in some segments of the LCx or some segments of the RCA, a problem of underestimating the FFR may occur. Accordingly, there is a need for the development of a technology for correcting the pressure difference value of a blood vessel according to the height difference of the blood vessel.

### SUMMARY

To solve the problem described above, the disclosure provides a method and an electronic device for correcting a pressure difference value of a blood vessel.

The disclosure may be implemented in various ways, including a method, a device (system) and/or a computer program stored in a computer-readable storage medium.

A method of correcting a pressure difference value of a blood vessel, performed by at least one processor, according to an embodiment, may include obtaining at least one image of a blood vessel of a subject captured by using an imaging apparatus, and imaging information of the imaging apparatus regarding the at least one image, obtaining, based on the imaging information, height information in a gravity direction of the blood vessel included in the at least one image, calculating, based on the height information, a height difference value between a first point and a second point of the blood vessel, calculating, based on the height difference value, a pressure difference value due to gravity between the first point and the second point of the blood vessel, and correcting, based on the pressure difference value due to gravity, the pressure difference value between the first point and the second point of the blood vessel.

According to an embodiment, the imaging information may include at least one of first distance information between an X-ray generator included in the imaging apparatus and an image acquisition apparatus included in the imaging apparatus, second distance information between the X-ray generator and the subject, position information of an examination table, pixel spacing information of the image acquisition apparatus, or angle information of the image acquisition apparatus with respect to the subject.

According to an embodiment, the obtaining of the height information may include obtaining, based on the imaging information, calibration information and imaging direction information, detecting a centerline of the blood vessel included in the at least one image, converting, based on the calibration information, the centerline into three-dimensional coordinates, and obtaining, based on the three-dimensional coordinates of the centerline, the height information in the gravity direction of the blood vessel.

According to an embodiment, the converting of the centerline into the three-dimensional coordinates may include converting, by using an intrinsic parameter included in the calibration information, image coordinates in which the centerline is represented into camera coordinates, wherein the intrinsic parameter includes a matrix generated based on the first distance information and the pixel spacing information, converting, by using an extrinsic parameter included in the calibration information, the camera coordinates into world coordinates, wherein the extrinsic parameter includes a rotation matrix generated based on the angle information, and a transition vector indicating a camera position determined based on the rotation matrix and the second distance information, and moving, by using the second distance information and position information of the X-ray generator, the world coordinates to coordinates having a rotational center of the imaging apparatus as a reference.

According to an embodiment, the at least one image may include a plurality of images obtained by capturing the blood vessel of the subject from different directions using the imaging apparatus, and the obtaining of the height information may include obtaining, based on imaging information of the imaging apparatus for each of the plurality of images, calibration information and imaging direction information, detecting a centerline of the blood vessel included in each of the plurality of images, reconstructing, based on the calibration information and the imaging information, the centerline of the blood vessel included in each of the plurality of images into a three-dimensional centerline, and obtaining, based on coordinates of the three-dimensional centerline, the height information in the gravity direction of the blood vessel.

According to an embodiment, the reconstructing of the centerline of the blood vessel included in each of the plurality of images into the three-dimensional centerline may include identifying, based on a first centerline of the blood vessel detected in a first image captured from a first direction of the imaging apparatus among the plurality of images, an epipolar line in a second image captured from a second direction of the imaging apparatus among the plurality of images, identifying, based on a second centerline of the blood vessel detected in the second image and the epipolar line, a common point of the first centerline and the second centerline, and generating the three-dimensional centerline through triangulation based on the common point.

According to an embodiment, the calculating of the pressure difference value may include calculating the pressure difference value due to gravity between the first point and the second point of the blood vessel by multiplying the height difference value by a blood density of the blood vessel and a gravitational acceleration.

According to an embodiment, the method of correcting a pressure difference value of a blood vessel may further include calculating information related to a blood flow characteristic based on the corrected pressure difference value.

According to an embodiment, the information related to the blood flow characteristic may include fractional flow reserve (FFR).

A computer program stored in a computer-readable storage medium may be provided to execute, on a computer, a method according to an embodiment.

According to an embodiment, an electronic device may include memory and at least one processor connected to the memory and configured to execute at least one computer-readable program included in the memory, wherein the at least one program may include instructions for obtaining at least one image of a blood vessel of a subject captured by using an imaging apparatus, and imaging information of the imaging apparatus regarding the at least one image, obtaining, based on the imaging information, height information in a gravity direction of the blood vessel included in the at least one image, calculating, based on the height information, a height difference value between a first point and a second point of the blood vessel, calculating, based on the height difference value, a pressure difference value due to gravity between the first point and the second point of the blood vessel, and correcting, based on the pressure difference value due to gravity, the pressure difference value between the first point and the second point of the blood vessel.

According to embodiments, more accurate FFR measurement may be supported by correcting a pressure difference value of a blood vessel based on a height difference of the blood vessel.

The effects of the disclosure are not limited to the effects mentioned above, and other effects not mentioned will be clearly understood by a person having ordinary skill in the art to which the disclosure pertains (referred to as an “ordinarily skilled person”) from the description of the claims.

### BRIEF DESCRIPTION OF THE DRAWINGS

The above and other aspects, features, and advantages of certain embodiments of the present disclosure will be more apparent from the following detailed description, taken in conjunction with the accompanying drawings, in which:

FIG. 1 is a diagram illustrating an electronic device for correcting a pressure difference value of a blood vessel according to an embodiment;

FIG. 2 is a diagram for describing a configuration of an electronic device according to an embodiment;

FIG. 3 is a diagram for describing a configuration of a processor of an electronic device according to an embodiment;

FIG. 4 is a diagram illustrating an imaging apparatus for capturing a blood vessel image according to an embodiment;

FIG. 5 is a diagram for describing angle information of an image acquisition apparatus with respect to a subject according to an embodiment;

FIG. 6 is a diagram for describing imaging information based on a positional relationship between an imaging apparatus and a subject according to an embodiment;

FIG. 7 is a diagram for explaining a method of obtaining height information in the gravity direction of a blood vessel from a blood vessel image according to an embodiment;

FIG. 8 is a diagram for explaining a method of obtaining height information in the gravity direction of a blood vessel from a plurality of blood vessel images according to an embodiment;

FIG. 9 is a diagram for explaining fractional flow reserve (FFR) in a blood vessel in which a pressure sensor is positioned higher than a blood vessel inlet according to an embodiment;

FIG. 10 is a diagram for explaining FFR in a blood vessel in which a pressure sensor is positioned lower than a blood vessel inlet according to an embodiment;

FIG. 11 is a diagram for explaining a method of correcting a pressure difference value of a blood vessel according to an embodiment; and

FIG. 12 is a diagram illustrating an artificial neural network model according to an embodiment.

### DETAILED DESCRIPTION

Hereinafter, specific details for carrying out the disclosure will be described in detail with reference to the accompanying drawings. However, in the following description, detailed descriptions of well-known functions or configurations that may unnecessarily obscure the subject matter of the disclosure will be omitted.

In the accompanying drawings, the same reference numerals are assigned to the same or corresponding components. In addition, in the description of the embodiments, a redundant description of the same or corresponding components may be omitted. However, even if a description of a component is omitted, such a component is not intended to be excluded from any embodiment.

The advantages and features of the disclosed embodiments and methods of achieving the advantages and features will be described more fully with reference to the accompanying drawings, in which embodiments of the disclosure are shown. The disclosure may, however, be embodied in many different forms and should not be construed as being limited to the embodiments set forth herein; rather these embodiments are provided so that the disclosure will be thorough and complete, and will fully convey the concept of the disclosure to one of ordinary skill in the art.

The terms used herein will be briefly described, and disclosed embodiments will be described in detail. The terms used in the embodiments are selected from among common terms that are currently widely used, in consideration of their function in the embodiments. However, the terms may become different according to an intention of one of ordinary skill in the art, a precedent, or the advent of new technology. In addition, in particular cases, the terms are discretionally selected by the applicant. In this instance, the meaning of those terms will be described in detail in the corresponding part of the detailed description. Therefore, the terms used in the embodiments are not merely designations of the terms, but the terms are defined based on the meaning of the terms and content throughout the embodiments.

It will be understood that the singular forms “a,” “an,” and “the” include plural referents unless the context clearly dictates otherwise. In addition, a plural form includes a singular form unless the context clearly dictates otherwise. It will be understood throughout the whole specification that, when one part “includes” or “comprises” one component, the part does not exclude other components but may further include other components, unless the context clearly dictates otherwise.

In addition, the terms “module” or “unit” as used in the disclosure refer to a software or hardware component, and a “module” or “unit” performs certain roles. However, the term “module” or “unit” is not limited to software or hardware. The “module” or “unit” may be configured to be in an addressable storage medium or configured to operate one or more processors. Thus, the “module” or “unit” may include at least one of, by way of example, components such as software components, object-oriented software components, class components, and task components, processes, functions, attributes, procedures, subroutines, segments of program code, drivers, firmware, microcode, circuitry, data, databases, data structures, tables, arrays, or variables. The functionalities provided in the components and “modules” or “units” may be combined into fewer components and “modules” or “units”, or may be further separated into additional components and “modules” or “units”.

According to an embodiment, a “module” or “unit” may include a processor and a memory. The “processor” should be interpreted broadly to include a general-purpose processor, a central processing unit (CPU), a microprocessor, a digital signal processor (DSP), a controller, a microcontroller, a state machine, etc. In some embodiments, the “processor” may indicate an application-specific integrated circuit (ASIC), a programmable logic device (PLD), a field programmable gate array (FPGA), etc. The term “processor” may refer to a combination of processing devices such as, for example, a combination of a DSP and a microprocessor, a combination of a plurality of microprocessors, a combination of one or more microprocessors in conjunction with a DSP core, or a combination of any other such configurations. In addition, the term “memory” should be interpreted broadly to include any electronic component capable of storing electronic information. The term “memory” may refer to various types of processor-readable media, such as random access memory (RAM), read-only memory (ROM), non-volatile random access memory (NVRAM), programmable read-only memory (PROM), erase-programmable read-only memory (EPROM), electrically erasable PROM (EEPROM), flash memory, a magnetic or optical data storage device, registers, and the like. When the processor can read information from a memory and/or write information to the memory, the memory is stated to be in an electronic communication state with the processor. The memory integrated in the processor is in an electronic communication state with the processor.

In addition, terms such as first, second, A, B, (a), and (b) used in the embodiments are used only to distinguish one component from another component, and the nature, order, or sequence of the corresponding component is not limited by the term.

In addition, in the following embodiments, when a component is described as being “connected,” “coupled,” or “linked” to another component, the component may be directly connected or linked to the other component, but it should be understood that another component may also be “connected,” “coupled,” or “linked” between the respective components.

In addition, the terms “comprises” and/or “comprising” used in the following embodiments specify the presence of the stated components, steps, operations, and/or elements, but do not exclude the presence or addition of one or more other components, steps, operations, and/or elements.

Hereinafter, various embodiments of the disclosure will be described in detail with reference to the accompanying drawings.

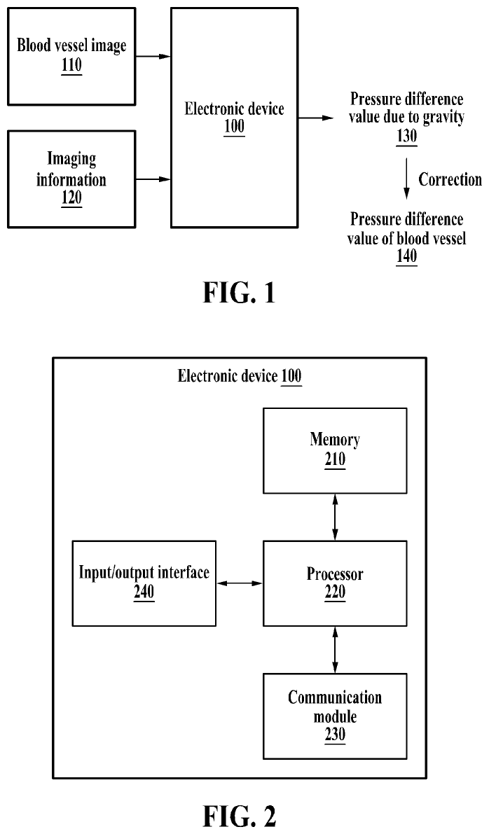

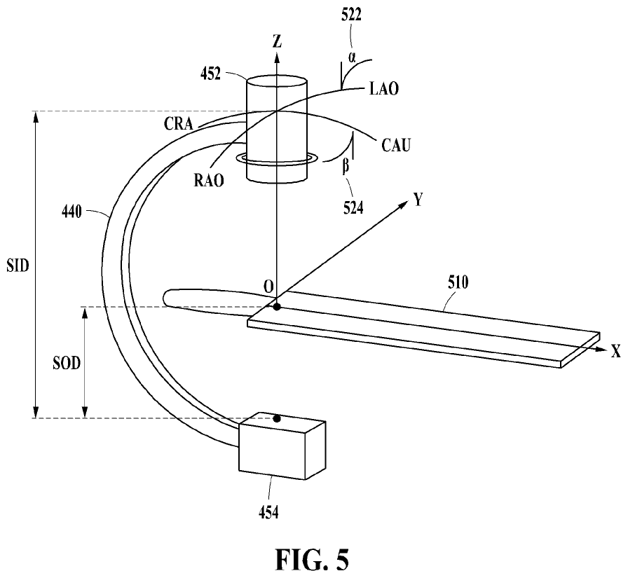

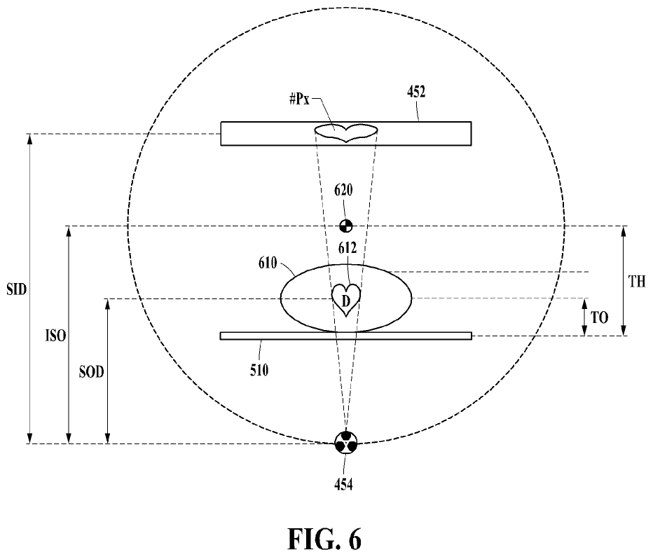

FIG. 1 is a diagram illustrating an electronic device for correcting a pressure difference value of a blood vessel according to an embodiment. Referring to FIG. 1, an electronic device 100 may correct a pressure difference value 140 of a blood vessel based on at least one blood vessel image 110 and imaging information 120 of an imaging apparatus for the at least one blood vessel image 110. Here, the blood vessel image 110 may refer to an image of a blood vessel of a subject (e.g., a patient) captured using an imaging apparatus for the purpose of diagnosing, treating, or preventing a disease. In addition, the blood vessel included in the blood vessel image 110 may include a coronary artery or a cerebral blood vessel. The coronary artery is positioned in a form that surrounds the heart in a crown shape, and may be classified into detailed types based on the position in which the coronary artery is arranged. For example, the coronary artery may be classified into the right coronary artery (RCA) that originates from the right side of the proximal portion of the ascending aorta and runs mainly to the right side of the heart, and the left coronary artery (LCA) that originates from the left side of the proximal portion of the ascending aorta and runs mainly to the left side of the heart. In addition, the LCA may be further classified into the left main coronary artery (LMCA) (hereinafter referred to as LM) originating from the upper left of the heart, the left anterior descending coronary artery (LAD) branching from the LM, and the left circumflex coronary artery (LCx). Cerebral blood vessels are positioned in a form extending from the aortic arch toward the head, and may be classified into detailed types based on the position in which the cerebral blood vessels are arranged. For example, the common carotid artery (CCA) branches into the internal carotid artery (ICA) and the external carotid artery (ECA), and the vertebral artery (VA) branches from the subclavian artery. A total of four blood vessels, i.e., the left and right ICAs and the left and right VAs, are the major blood vessels that supply blood to the brain. The four blood vessels may be interconnected via the anterior communicating artery (ACoA) and the left and right posterior communicating arteries (PCoA), and because the four blood vessels form a single ring (circle), the structure is referred to as the circle of Willis. According to an embodiment, the blood vessel image 110 may include an image captured through coronary angiography (CAG). For example, the blood vessel image 110 may include an image of the coronary artery of a subject captured using an imaging apparatus (e.g., a C-arm X-ray imaging apparatus) while a contrast agent is injected into the blood vessel of the subject. In addition, the imaging information 120 of the imaging apparatus for the blood vessel image 110 is the imaging information 120 of the imaging apparatus at the time the blood vessel image 110 was captured. The imaging information 120 may include at least one of distance information between an X-ray generator included in the imaging apparatus and an image acquisition apparatus included in the imaging apparatus, distance information between the X-ray generator and the subject, position information of an examination table, pixel spacing information of the image acquisition apparatus, or angle information of the image acquisition apparatus with respect to the subject. The imaging information 120 of the imaging apparatus for the blood vessel image 110 will be described in detail with reference to FIGS. 5 and 6.

Although a storage system capable of communicating with the electronic device 100 is not shown in FIG. 1, the electronic device 100 may be configured to be connected to or capable of communicating with one or more storage systems. A storage system configured to be connected to or capable of communicating with the electronic device 100 may include a device or a cloud system that stores and manages various data associated with the operation of correcting the pressure difference value 140 of the blood vessel using the blood vessel image 110 and the imaging information 120 of the imaging apparatus for the blood vessel image 110. For efficient management of data, the storage system may store and manage various data using a database. Here, the various data may include any data associated with the operation of correcting the pressure difference value 140 of the blood vessel using the blood vessel image 110 and the imaging information 120 of the imaging apparatus for the blood vessel image 110. For example, the various data may include, but are not limited to, a machine learning model, training data, the blood vessel image 110, and the imaging information 120 associated with the operation of correcting the pressure difference value 140 of the blood vessel using the blood vessel image 110 and the imaging information 120 of the imaging apparatus for the blood vessel image 110.

According to an embodiment, after the blood vessel of the subject is captured through the imaging apparatus, the blood vessel image 110 in which the blood vessel is captured may be input to the electronic device 100. For example, the electronic device 100 may be connected to the imaging apparatus via wired or wireless communication. The blood vessel image 110 may be provided from the imaging apparatus to the electronic device 100 through a communication module. In an embodiment, the electronic device 100 may be provided integrally with the imaging apparatus. In another example, the electronic device 100 may receive the blood vessel image 110 from an external electronic device (e.g., an external storage device) connected to the electronic device 100 through a communication module. The manner in which the electronic device 100 obtains the blood vessel image 110 is not limited to the examples described above, and any manner may be used.

According to an embodiment, the imaging information 120 of the imaging apparatus for the blood vessel image 110 may be input to the electronic device 100 together with the blood vessel image 110 or at a certain time interval. For example, when the electronic device 100 is connected to the imaging apparatus via wired or wireless communication, the imaging apparatus may obtain the imaging information 120 of the imaging apparatus at the time of capturing the blood vessel image 110, and may provide the imaging information 120 obtained together with the blood vessel image 110 to the electronic device 100 through a communication module. In another example, when the electronic device 100 is provided integrally with the imaging apparatus, the electronic device 100 may obtain the imaging information 120 of the imaging apparatus together with the blood vessel image 110 at the time the imaging apparatus captures the blood vessel image 110. In yet another example, the electronic device 100 may receive the imaging information 120 together with the blood vessel image 110 from an external electronic device (e.g., an external storage device) connected to the electronic device 100 through a communication module.

To describe the process of correcting the pressure difference value 140 of the blood vessel based on the blood vessel image 110 and the imaging information 120 of the imaging apparatus for the blood vessel image 110, the electronic device 100 may first obtain at least one blood vessel image 110 and the imaging information 120 of the imaging apparatus for the at least one blood vessel image 110. For example, the electronic device 100 may receive at least one blood vessel image 110 and the imaging information 120 of the imaging apparatus for the at least one blood vessel image 110 through a communicatively accessible storage medium (e.g., a hospital system, a local/cloud storage system, or the like).

The electronic device 100 may then, based on the imaging information 120, obtain height information in the gravity direction of the blood vessel included in the at least one blood vessel image 110. According to an embodiment, the electronic device 100 may obtain calibration information and imaging direction information based on the imaging information 120, detect a centerline of the blood vessel included in the at least one blood vessel image 110, convert the centerline into three-dimensional coordinates based on the calibration information, and obtain height information in the gravity direction of the blood vessel based on the three-dimensional coordinates of the centerline. Here, to convert the centerline into three-dimensional coordinates, the electronic device 100 may convert, by using an intrinsic parameter included in the calibration information, image coordinates in which the centerline is represented into camera coordinates, where the intrinsic parameter includes a matrix generated based on distance information between the X-ray generator and the image acquisition apparatus and pixel spacing information of the image acquisition apparatus, may convert, by using an extrinsic parameter included in the calibration information, the camera coordinates into world coordinates, where the extrinsic parameter includes a rotation matrix generated based on the angle information of the image acquisition apparatus with respect to the subject and a transition vector indicating a camera position determined based on the rotation matrix and distance information between the X-ray generator and the subject, and may move, by using the distance information between the X-ray generator and the subject and position information of the X-ray generator, the world coordinates to coordinates having the rotational center of the imaging apparatus as a reference.

According to an embodiment, when the at least one blood vessel image 110 includes a plurality of blood vessel images 110 captured from different directions of the blood vessel of the subject using the imaging apparatus, the electronic device 100 may obtain, based on the imaging information 120 of the imaging apparatus for each of the plurality of blood vessel images 110, calibration information and imaging direction information, may detect a centerline of the blood vessel included in each of the plurality of blood vessel images 110, may reconstruct, based on the calibration information and the imaging information 120, the centerline of the blood vessel included in each of the plurality of blood vessel images 110 into a three-dimensional centerline, and may obtain, based on coordinates of the three-dimensional centerline, the height information in the gravity direction of the blood vessel. Here, to reconstruct the centerline of the blood vessel included in each of the plurality of blood vessel images 110 into the three-dimensional centerline, the electronic device 100 may identify, based on a first centerline of the blood vessel detected in a first image captured from a first direction of the imaging apparatus among the plurality of blood vessel images 110, an epipolar line in a second image captured from a second direction of the imaging apparatus among the plurality of blood vessel images 110, may identify, based on a second centerline of the blood vessel detected in the second image and the epipolar line, a common point (e.g., a common image point (CIP)) of the first centerline and the second centerline, and may generate the three-dimensional centerline through triangulation based on the common point.

The electronic device 100 may then calculate a height difference value between a first point and a second point of the blood vessel based on the height information.

The electronic device 100 may then calculate a pressure difference value 130 due to gravity (e.g., hydrostatic pressure) between the first point and the second point of the blood vessel based on the height difference value. For example, the electronic device 100 may calculate the pressure difference value 130 due to gravity between the first point and the second point of the blood vessel by multiplying the height difference value by the blood density of the blood vessel and a gravitational acceleration.

The electronic device 100 may then correct the pressure difference value 140 between the first point and the second point of the blood vessel based on the pressure difference value 130 due to gravity. For example, the electronic device 100 may calculate the pressure difference value 140 between the first point and the second point of the blood vessel by adding the pressure difference value 130 due to gravity to the pressure difference value due to the narrowing of the inner diameter of the lesion. Here, the pressure difference value due to the narrowing of the inner diameter of the lesion may be a value measured using fractional flow reserve (FFR) equipment.

FIG. 2 is a diagram for describing a configuration of an electronic device according to an embodiment. Referring to FIG. 2, an electronic device 100 may include a memory 210, a processor 220, a communication module 230, and an input/output interface 240. However, the configuration of the electronic device 100 is not limited thereto. According to various embodiments, the electronic device 100 may omit at least one of the above-described components, and may further include at least one other component. For example, the electronic device 100 may further include a display. In this case, the electronic device 100 may display, on the display, a blood vessel image obtained by capturing a blood vessel (e.g., the blood vessel image 110 of FIG. 1).

The memory 210 may store various types of data used by at least one other component (e.g., the processor 220) of the electronic device 100. The data may include, for example, software (or a program) and input data or output data for a command related thereto.

The memory 210 may include any non-transitory computer-readable storage medium. According to an embodiment, the memory 210 may include a permanent mass storage device such as a disk drive, a solid-state drive (SSD), or flash memory. In another example, a permanent mass storage device such as a ROM, an SSD, flash memory, or a disk drive may be included in the electronic device 100 as a separate permanent storage device distinct from the memory 210. In addition, the memory 210 may store an operating system and at least one piece of program code (e.g., instructions such as those for correcting a pressure difference value of a blood vessel that are installed and executed by the electronic device 100). In FIG. 2, the memory 210 is illustrated as a single memory. However, this is only for convenience of description, and the memory 210 may include a plurality of memories and/or buffer memories.

Software components may be loaded from a computer-readable storage medium separate from the memory 210. Such a separate computer-readable storage medium may include a storage medium directly connectable to the electronic device 100, and may include, for example, computer-readable storage media such as a floppy drive, a disk, a tape, a digital versatile disc (DVD)/compact disc (CD)-ROM drive, and a memory card. In another example, software components may be loaded into the memory 210 through the communication module 230 rather than through a computer-readable storage medium. For example, at least one program may be loaded into the memory 210 based on a computer program (e.g., a program for transmitting data such as a blood vessel image in which a blood vessel has been captured) that is installed by files provided through the communication module 230 by developers or a file distribution system that distributes installation files of an application.

The processor 220 may execute software (or a program) to control at least one other component (e.g., a hardware or software component) of the electronic device 100 connected to the processor 220, and may perform various data processing or computation. According to an embodiment, as at least a part of data processing or computation, the processor 220 may load instructions or data received from another component (e.g., the communication module 230) into a volatile memory, process the instructions or data stored in the volatile memory, and store result data in a non-volatile memory.

The processor 220 may be configured to process commands of a computer program by performing basic arithmetic, logic, and input/output operations. The commands may be provided to the electronic device 100 or another external system by the memory 210 or the communication module 230. For example, the processor 220 may correct a pressure difference value of a blood vessel using a blood vessel image and imaging information of an imaging apparatus for the blood vessel image. The processor 220 may then store the corrected pressure difference value of the blood vessel in the memory 210, display the corrected pressure difference value on a display of the electronic device 100, or transmit the corrected pressure difference value to an external electronic device via the communication module 230. Alternatively, the processor 220 may perform additional analysis operations, such as calculating information related to a blood flow characteristic, using the pressure difference value of the blood vessel. Here, the information related to a blood flow characteristic may include FFR. For example, the processor 220 may calculate FFR using the pressure difference value of the blood vessel. In FIG. 2, the processor 220 is illustrated as a single processor; however, this is for convenience of description only, and the processor 220 may include a plurality of processors.

The communication module 230 may support establishing a direct (e.g., wired) communication channel or wireless communication channel between the electronic device 100 and an external electronic device and performing communication through the established communication channel. For example, the communication module 230 may provide a configuration or function for the electronic device 100 and an external electronic device (e.g., a user terminal or a cloud server) to communicate with each other over a network. For example, control signals, commands, data, and the like provided under the control of the processor 220 of the electronic device 100 may be transmitted to an external electronic device through the communication module 230, a network, and a communication module of the external electronic device. For example, the electronic device 100 may receive, from an external electronic device via the communication module 230, a blood vessel image obtained by capturing a blood vessel of a subject.

The input/output interface 240 may be a means for interfacing with an input or output device (not shown) that is connected to the electronic device 100 or that the electronic device 100 may include. For example, the input/output interface 240 may include at least one of a peripheral component interconnect (PCI) express interface and an Ethernet interface. In FIG. 2, the input/output interface 240 is illustrated as a component configured separately from the processor 220; however, the disclosure is not limited thereto, and the input/output interface 240 may be configured to be included in the processor 220.

According to an embodiment, the processor 220 may perform functions related to correcting a pressure difference value of a blood vessel based on a blood vessel image and imaging information of an imaging apparatus for the blood vessel image. The processor 220 may execute at least one computer-readable program included in the memory 210 to perform functions related to correcting the pressure difference value of the blood vessel. Here, the at least one program may include instructions for obtaining at least one image of a blood vessel of a subject captured by using an imaging apparatus, and imaging information of the imaging apparatus regarding the at least one image, obtaining, based on the imaging information, height information in the gravity direction of the blood vessel included in the at least one image, calculating, based on the height information, a height difference value between a first point and a second point of the blood vessel, calculating, based on the height difference value, a pressure difference value due to gravity between the first point and the second point of the blood vessel, and correcting, based on the pressure difference value due to gravity, the pressure difference value between the first point and the second point of the blood vessel. In the following description, for convenience of explanation, the execution of at least one program by the processor 220 to perform functions related to correcting the pressure difference value of the blood vessel based on a blood vessel image and imaging information of an imaging apparatus for the blood vessel image may be described as the processor 220 performing functions related to correcting the pressure difference value of the blood vessel based on the blood vessel image and the imaging information of the imaging apparatus for the blood vessel image. For example, the at least one program including instructions related to correcting the pressure difference value of the blood vessel based on a blood vessel image and imaging information of an imaging apparatus for the blood vessel image may be described as corresponding to the processor 220 performing functions related to correcting the pressure difference value of the blood vessel based on the blood vessel image and the imaging information of the imaging apparatus for the blood vessel image.

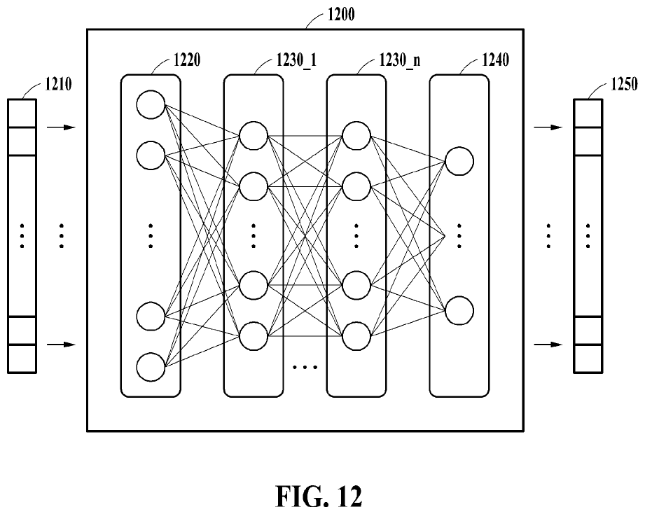

According to an embodiment, the processor 220 may correct the pressure difference value of the blood vessel through a machine learning model that takes a blood vessel image and imaging information of an imaging apparatus for the blood vessel image as input. Here, the machine learning model may include any model used to infer an answer to a given input. According to an embodiment, the machine learning model may include an artificial neural network model including an input layer, a plurality of hidden layers, and an output layer. Here, each layer may include one or more nodes. In addition, the machine learning model may include weights associated with a plurality of nodes included in the machine learning model. Here, the weights may include arbitrary parameters associated with the machine learning model. The machine learning model of the disclosure may be a model trained using various training methods. For example, various training methods, such as supervised learning, semi-supervised learning, unsupervised learning (or autonomous learning), and reinforcement learning, may be used in the disclosure. In the disclosure, a machine learning model may refer to an artificial neural network model, and an artificial neural network model may refer to a machine learning model. The artificial neural network model will be described in detail with reference to FIG. 12.

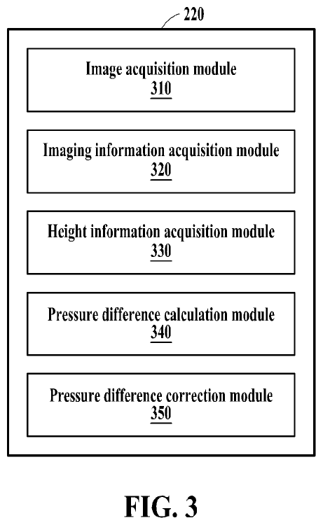

FIG. 3 is a diagram for describing a configuration of a processor of an electronic device according to an embodiment. Referring to FIG. 3, a processor 220 may use a blood vessel image (e.g., the blood vessel image 110 of FIG. 1) and imaging information of an imaging apparatus for the blood vessel image (e.g., the imaging information 120 of FIG. 1) to calculate a pressure difference value due to gravity between a first point and a second point of the blood vessel (e.g., the pressure difference value 130 due to gravity of FIG. 1), and may correct a pressure difference value between the first point and the second point of the blood vessel (e.g., the pressure difference value 140 of FIG. 1) based on the pressure difference value due to gravity. To this end, the processor 220 may include an image acquisition module 310, an imaging information acquisition module 320, a height information acquisition module 330, a pressure difference calculation module 340, and a pressure difference correction module 350. However, the types of components included in the processor 220 are classified according to functions related to correcting the pressure difference value of a blood vessel using a blood vessel image and imaging information of an imaging apparatus for a blood vessel image, and the types and number of components are not limited thereto. In addition, at least one of the components included in the processor 220 may be implemented in the form of instructions stored in a memory (e.g., the memory 210 of FIG. 2).

The image acquisition module 310 may obtain at least one blood vessel image by capturing a blood vessel. According to an embodiment, the image acquisition module 310 may obtain an image of a blood vessel of a subject captured using an imaging apparatus (e.g., a C-arm X-ray imaging apparatus) while a contrast agent is injected into the blood vessel of the subject. For example, a blood vessel image may include an image captured through CAG. Such a blood vessel image may be received from a storage system connected to or in communication with the electronic device 100 (e.g., a hospital system, an electronic medical record, a prescription delivery system, a medical imaging system, a laboratory information system, a local/cloud storage system, or the like), an internal memory, and/or a user terminal.

The imaging information acquisition module 320 may obtain imaging information of an imaging apparatus for at least one blood vessel image. Here, the imaging apparatus may include, for example, a C-arm X-ray imaging apparatus. In addition, the imaging information is imaging information of the imaging apparatus at the time the at least one blood vessel image was captured. The imaging information may include at least one of distance information between an X-ray generator included in the imaging apparatus and an image acquisition apparatus included in the imaging apparatus, distance information between the X-ray generator and a subject, position information of an examination table, pixel spacing information of the image acquisition apparatus, or angle information of the image acquisition apparatus with respect to the subject.

The height information acquisition module 330 may obtain, based on the imaging information, height information in the gravity direction of a blood vessel included in the at least one blood vessel image. When the number of blood vessel images used to obtain the height information is one, a method of converting two-dimensional coordinates of the blood vessel image into three-dimensional coordinates may be used. In addition, when the number of blood vessel images used to obtain the height information is plural, a method of converting two-dimensional coordinates of the blood vessel images into three-dimensional coordinates or a method of reconstructing a three-dimensional shape of the blood vessel using the plurality of blood vessel images may be used.

With respect to the method of converting two-dimensional coordinates of a blood vessel image into three-dimensional coordinates, the height information acquisition module 330 may obtain calibration information and imaging direction information based on the imaging information. Then, the height information acquisition module 330 may detect a centerline of the blood vessel included in the at least one blood vessel image. Then, the height information acquisition module 330 may convert the centerline into three-dimensional coordinates based on the calibration information. For example, the height information acquisition module 330 may convert image coordinates in which the centerline is represented into camera coordinates, using an intrinsic parameter included in the calibration information. In this case, the intrinsic parameter may include a matrix generated based on distance information between the X-ray generator and the image acquisition apparatus and pixel spacing information of the image acquisition apparatus. In addition, the height information acquisition module 330 may convert the camera coordinates into world coordinates using an extrinsic parameter included in the calibration information. In this case, the extrinsic parameter may include a rotation matrix generated based on angle information of the image acquisition apparatus with respect to the subject and a transition vector indicating a camera position determined based on the rotation matrix and distance information between the X-ray generator and the subject. In addition, the height information acquisition module 330 may move the world coordinates to coordinates having the rotational center of the imaging apparatus as a reference, using the distance information between the X-ray generator and the subject and the position information of the X-ray generator. The height information acquisition module 330 may then obtain height information in the gravity direction of the blood vessel based on the three-dimensional coordinates of the centerline.

With regard to the method of reconstructing a three-dimensional shape of a blood vessel using a plurality of blood vessel images, at least one blood vessel image may include a plurality of blood vessel images obtained by capturing the blood vessel of the subject from different directions using the imaging apparatus. In this case, the height information acquisition module 330 may obtain calibration information and imaging direction information based on the imaging information of the imaging apparatus for each of the plurality of blood vessel images. The height information acquisition module 330 may then detect the centerline of the blood vessel included in each of the plurality of blood vessel images. The height information acquisition module 330 may then reconstruct the centerline of the blood vessel included in each of the plurality of blood vessel images into a three-dimensional centerline, based on the calibration information and the imaging information. For example, the height information acquisition module 330 may identify, based on a first centerline of the blood vessel detected in a first image captured from a first direction of the imaging apparatus among the plurality of blood vessel images, an epipolar line in a second image captured from a second direction of the imaging apparatus among the plurality of blood vessel images. In addition, the height information acquisition module 330 may, based on the second centerline of the blood vessel detected in the second image and the epipolar line, identify a common point of the first centerline and the second centerline. In addition, the height information acquisition module 330 may generate a three-dimensional centerline through triangulation based on the common point. The height information acquisition module 330 may then obtain height information in the gravity direction of the blood vessel based on the coordinates of the three-dimensional centerline.

The height information acquisition module 330 may calculate a height difference value at two points of the blood vessel based on the acquisition of height information in the gravity direction of the blood vessel. For example, the height information acquisition module 330 may calculate a height difference value between the first point and the second point of a blood vessel.

The pressure difference calculation module 340 may calculate a pressure difference value due to gravity (e.g., hydrostatic pressure) at two points of a blood vessel, based on the height difference value at the two points of the blood vessel. For example, the pressure difference calculation module 340 may calculate a pressure difference value due to gravity between a first point and a second point of a blood vessel by multiplying the height difference value between the first point and the second point of the blood vessel by the blood density of the blood vessel and the gravitational acceleration.

The pressure difference correction module 350 may correct the pressure difference value between the two points of the blood vessel, based on the pressure difference value due to gravity at the two points of the blood vessel. For example, the pressure difference correction module 350 may calculate the pressure difference value between the two points (e.g., the first point and the second point) of the blood vessel by adding the pressure difference value due to gravity to the pressure difference value due to the narrowing of the inner diameter of a lesion. Here, the pressure difference value due to the narrowing of the inner diameter of the lesion may be a value measured using FFR equipment.

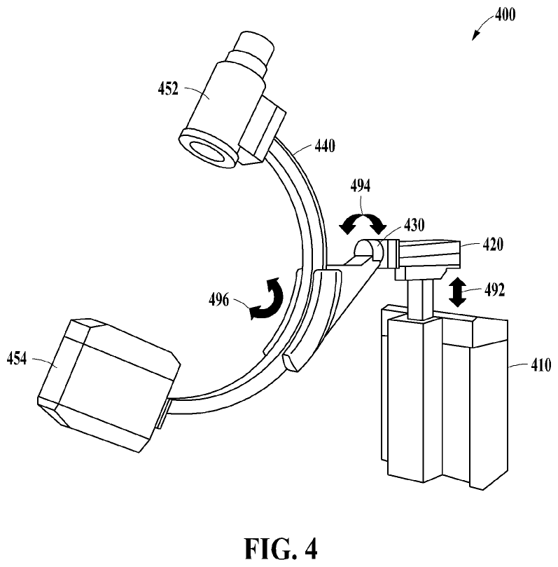

FIG. 4 is a diagram illustrating an imaging apparatus for capturing a blood vessel image according to an embodiment. Referring to FIG. 4, an imaging apparatus 400 may capture a blood vessel of a subject. For example, the imaging apparatus 400 may capture a blood vessel of a subject while a contrast agent is injected into the blood vessel of the subject. According to an embodiment, the imaging apparatus 400 may include a C-arm X-ray imaging apparatus.

The imaging apparatus 400 may include a main body 410, an elevating unit 420, a rotating unit 430, a C-type frame unit 440, an X-ray generator 454, and an image acquisition apparatus 452. However, the configuration of the imaging apparatus 400 is not limited thereto. The imaging apparatus 400 may be provided in any configuration and in any form, as long as the configuration includes the image acquisition apparatus 452.

The main body 410 may have an elevation driver built therein. According to an embodiment, the main body 410 may have a controller (e.g., a processor) built therein for controlling the configuration of the imaging apparatus 400. In addition, when the imaging apparatus 400 is connected to the electronic device 100 of FIGS. 1 and 2 via a communication module, the communication module may be built into the main body 410.

The elevating unit 420 may be fixed to the upper end of the elevation driver built into the main body 410, and may be elevated in a first direction 492 (e.g., an up-down direction). For example, the elevating unit 420 may be adjusted to match the height of an examination target (or imaging target) (e.g., the heart) according to the posture of the subject.

The rotating unit 430 may have one end connected to the elevating unit 420 so as to be rotatable 494 about a first axis in a second direction perpendicular to the first direction 492, and may have a curved surface formed at the other end. When the rotating unit 430 rotates 494 about the first axis, a rotation angle (e.g., a first rotation angle) of the image acquisition apparatus 452 may be changed. Here, the first axis may be an axis in the up-down direction (or longitudinal direction) of the subject (e.g., a direction connecting the head to the feet) or an axis parallel thereto. For example, rotation 494 of the rotating unit 430 about the first axis indicates that the image acquisition apparatus 452 rotates about the first axis. In addition, rotation of the image acquisition apparatus 452 about the first axis may indicate that the image acquisition apparatus 452 rotates in the left-right direction about the torso (or heart) of the subject. In the following description, rotation angle information of the image acquisition apparatus 452 formed when the rotating unit 430 rotates 494 about the first axis may be referred to as first rotation angle information.

The C-type frame unit 440 may be slidably 496 connected to a curved surface formed on the rotating unit 430, and may be provided in a C-shape (or a ring shape with a portion cut away). Due to the shape of the C-type frame unit 440, the imaging apparatus 400 may be referred to as a C-arm or a C-arm imaging apparatus. When the C-type frame unit 440 slides 496 on the curved surface formed on the rotating unit 430, the rotation angle (e.g., a second rotation angle) of the image acquisition apparatus 452 may be changed. When the C-type frame unit 440 slides 496 on the curved surface formed on the rotating unit 430, the image acquisition apparatus 452 may rotate about a second axis in a third direction perpendicular to the first direction 492 and the second direction. Here, the second axis may be an axis in the left-right direction (or width direction) of the subject (e.g., a direction connecting both shoulders or both arms) or an axis parallel thereto. For example, the sliding 496 of the C-type frame unit 440 on the curved surface formed on the rotating unit 430 indicates that the image acquisition apparatus 452 rotates about the second axis. In addition, rotation of the image acquisition apparatus 452 about the second axis may indicate that the image acquisition apparatus 452 rotates in the up-down direction about the torso (or heart) of the subject. In the following description, rotation angle information of the image acquisition apparatus 452 formed when the C-type frame unit 440 slides 496 on the curved surface formed on the rotating unit 430 may be referred to as second rotation angle information.

The X-ray generator 454 may be disposed at one end of the C-type frame unit 440. The image acquisition apparatus 452 may be disposed at the other end of the C-type frame unit 440. The X-ray generator 454 may generate X-rays and transmit the X-rays through an examination target (or imaging target). The image acquisition apparatus 452 may detect the amount of transmitted X-rays and perform signal processing thereon to obtain an image (e.g., the blood vessel image 110 of FIG. 1). At this time, the imaging apparatus 400 may obtain, together with the image obtained through the image acquisition apparatus 452, imaging information of the imaging apparatus 400 at the time of capturing the image (e.g., the imaging information 120 of FIG. 1). The imaging information may include, for example, at least one of distance information between the X-ray generator 454 and the image acquisition apparatus 452, distance information between the X-ray generator 454 and the subject, position information of an examination table, pixel spacing information of the image acquisition apparatus 452, or angle information of the image acquisition apparatus 452 with respect to the subject (e.g., the first rotation angle information and the second rotation angle information). FIG. 5 is a diagram for describing angle information of an image acquisition apparatus with respect to a subject according to an embodiment. Referring to FIG. 5, the imaging apparatus 400 may change (or set) angles 522 and 524 of the image acquisition apparatus 452 to correspond to a blood vessel region to be imaged, in order to identify in which region an abnormality has occurred with respect to the blood vessel of the subject. As described with reference to FIG. 4, in the imaging apparatus 400, the X-ray generator 454 and the image acquisition apparatus 452 are disposed at both ends of the C-type frame unit 440. The angles 522 and 524 of the image acquisition apparatus 452 may be changed by the C-type frame unit 440 sliding (e.g., the sliding 496 of FIG. 4) on a curved surface of a rotating unit (e.g., the rotating unit 430 of FIG. 4) or by the rotating unit rotating (e.g., the rotation 494 of FIG. 4).

The angles 522 and 524 of the image acquisition apparatus 452 may be set with reference to the examination target (or imaging target). For example, when the subject lies on an examination table 510, the up-down direction (or longitudinal direction) of the subject (e.g., the direction connecting the head to the feet) may be the X-axis direction. The left-right direction (or width direction) of the subject (e.g., the direction connecting two shoulders or two arms to each other) may be the Y-axis direction. More specifically, the direction from the head toward the feet may be the (+)X-axis direction. The direction from the feet toward the head may be the (−)X-axis direction. The direction from the right shoulder (or right arm) toward the left shoulder (or left arm) may be the (+)Y-axis direction. The direction from the left shoulder (or left arm) toward the right shoulder (or right arm) may be the (−)Y-axis direction. At this time, the first rotation angle information of the image acquisition apparatus 452 formed when the rotating unit rotates and the image acquisition apparatus 452 rotates about the X-axis may be set as a first rotation angle (α) 522. The second rotation angle information of the image acquisition apparatus 452 formed when the C-type frame unit 440 slides along the curved surface of the rotating unit and the image acquisition apparatus 452 rotates about the Y-axis may be set as a second rotation angle (β) 524.

The first rotation angle 522 may be referred to as a primary angle. When the first rotation angle 522 has a rotation angle in the (−)Y-axis direction, the image may be referred to as having a right anterior oblique (RAO) view, and the first rotation angle 522 may be expressed as an RAO angle. In addition, when the first rotation angle 522 has a rotation angle in the (+)Y-axis direction, the image may be referred to as having a left anterior oblique (LAO) view, and the first rotation angle 522 may be expressed as an LAO angle. In addition, when the first rotation angle 522 has a rotation angle centered on the Y-axis (i.e., 0 degrees), the image may be referred to as having an anteroposterior (AP) view.

The second rotation angle 524 may be referred to as a secondary angle. When the second rotation angle 524 is a rotation angle in the (−)X-axis direction, the image may be referred to as having a cranial (CRA) (or CRANIAL) view, and the second rotation angle 524 may be expressed as a CRA angle. In addition, when the second rotation angle 524 is a rotation angle in the (+)X-axis direction, the image may be referred to as having a caudal (CAU) (or CAUDAL) view, and the second rotation angle 524 may be expressed as a CAU angle.

According to an embodiment, through a combination of the first rotation angle 522 and the second rotation angle 524, the type of blood vessel to be identified in the image or to be included in the image may be set.

FIG. 6 is a diagram for describing imaging information based on a positional relationship between an imaging apparatus and a subject according to an embodiment. Referring to FIG. 6, a processor (e.g., the processor 220 of FIGS. 2 and 3) of an electronic device (e.g., the electronic device 100 of FIGS. 1 and 2) for correcting a pressure difference value of a blood vessel may obtain imaging information (e.g., the imaging information 120 of FIG. 1) of an imaging apparatus 400 for at least one blood vessel image, together with at least one blood vessel image (e.g., the blood vessel image 110 of FIG. 1) obtained by capturing the blood vessel. The imaging information may include at least one of distance information between the X-ray generator 454 and the image acquisition apparatus 452 (source to image receptor distance (SID)), distance information between the X-ray generator 454 and a subject 610 (or an examination target 612 (e.g., the heart)) (source to object distance (SOD)), position information of the examination table 510, pixel spacing information of the image acquisition apparatus 452, or angle information of the image acquisition apparatus 452 with respect to the subject 610 (e.g., first rotation angle information and second rotation angle information).

According to an embodiment, the processor may calculate the distance information between the X-ray generator 454 and the subject 610 using Equation 1 below.

SOD=ISO−(TH−TO) [Equation 1]

Here, SOD represents the distance information between the X-ray generator 454 and the subject 610 (or the examination target 612 (e.g., the heart)); ISO represents the distance information between the X-ray generator 454 and a rotational center 620 of the imaging apparatus 400; TH represents the distance information between the examination table 510 and the rotational center 620 of the imaging apparatus 400; and TO represents the distance information between the examination table 510 and the subject 610 (or the examination target 612 (e.g., the heart)).

According to an embodiment, the processor may obtain a direction vector from the X-ray generator 454 to the coordinates indicating the subject 610 (or the examination target 612 (e.g., the heart)) using Equation 2 below.

$$D = \# Px * Δ Px * \frac{SOD}{SID} \qquad [ Equation 2 ]$$

Here, D represents the direction vector from the X-ray generator 454 to the coordinates indicating the subject 610 (or the examination target 612 (e.g., the heart)); #Px represents the number of pixels of the image acquisition apparatus 452; ΔPx represents the pixel spacing information of the image acquisition apparatus 452; SOD represents the distance information between the X-ray generator 454 and the subject 610 (or the examination target 612 (e.g., the heart)); and SID represents the distance information between the X-ray generator 454 and the image acquisition apparatus 452.

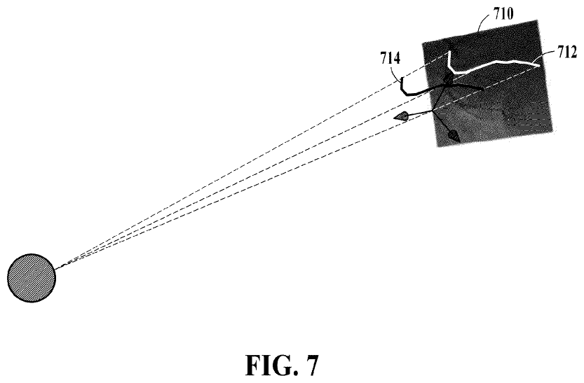

FIG. 7 is a diagram for explaining a method of obtaining height information in the gravity direction of a blood vessel from a blood vessel image according to an embodiment. With reference to FIG. 7, a processor (e.g., the processor 220 of FIGS. 2 and 3) of an electronic device (e.g., the electronic device 100 of FIGS. 1 and 2) for correcting a pressure difference value of a blood vessel may obtain height information in the gravity direction of the blood vessel included in at least one blood vessel image 710, based on imaging information (e.g., the imaging information 120 of FIG. 1) of an imaging apparatus (e.g., the imaging apparatus 400 of FIG. 4) with respect to the at least one blood vessel image 710 (e.g., the blood vessel image 110 of FIG. 1).

The processor may obtain calibration information and imaging direction information based on the imaging information. The processor may then detect a centerline 712 of the blood vessel included in the at least one blood vessel image 710. The processor may then convert the centerline 712 of the blood vessel into a centerline 714 of the blood vessel having three-dimensional coordinates, based on the calibration information. For example, the processor may convert image coordinates in which the centerline 712 of the blood vessel is represented into camera coordinates, using an intrinsic parameter included in the calibration information. In this case, the intrinsic parameter may include a matrix generated based on distance information between an X-ray generator (e.g., the X-ray generator 454 of FIGS. 4 to 6) and an image acquisition apparatus (e.g., the image acquisition apparatus 452 of FIGS. 4 to 6) and pixel spacing information of the image acquisition apparatus. In addition, the processor may convert the camera coordinates to world coordinates using an extrinsic parameter included in the calibration information. In this case, the extrinsic parameter may include a rotation matrix generated based on the angle information of the image acquisition apparatus with respect to a subject (e.g., the subject 610 of FIG. 4) and a transition vector indicating a camera position determined based on the rotation matrix and the distance information between the X-ray generator and the subject.

According to an embodiment, the processor may convert the centerline 712 of the blood vessel having two-dimensional coordinates included in the blood vessel image 710 into the centerline 714 of the blood vessel having three-dimensional coordinates through Equation 3, Equation 4, and Equation 5 below.

$$[ \begin{matrix}X \\ Y \\ Z\end{matrix} ] = R ( {K}^{- 1} [ \begin{matrix}x \\ y \\ 1\end{matrix} ] ) + T \qquad [ Equation 3 ]$$

$$K = [ \begin{matrix}\frac{SID}{IPS} & 0 & Cx \\ 0 & \frac{SID}{IPS} & Cy \\ 0 & 0 & 1\end{matrix} ] \qquad [ Equation 4 ]$$

$$T = R * [ \begin{matrix}0 \\ 0 \\ - 1\end{matrix} ] * S O D \qquad [ Equation 5 ]$$

Here, (X, Y, Z) may denote the three-dimensional coordinates of the blood vessel centerline 714; (x, y) may denote the two-dimensional coordinates of the blood vessel centerline 712; K may denote the intrinsic parameter; R may denote the rotation matrix generated based on the angle information of the image acquisition apparatus with respect to the subject; T may denote the transition vector indicating a camera position determined based on R and SOD; SOD may denote the distance information between the X-ray generator and the subject (or examination target); SID may denote the distance information between the X-ray generator and the image acquisition apparatus; and IPS may denote the pixel spacing information of the image acquisition apparatus.

The processor may then shift the world coordinates to coordinates having the rotational center of the imaging apparatus as a reference (e.g., the rotational center 620 of the imaging apparatus of FIG. 6), using the distance information between the X-ray generator and the subject and the position information of the X-ray generator. The processor may then obtain the height information in the gravity direction of the blood vessel based on the three-dimensional coordinates of the centerline 714 of the blood vessel.

According to an embodiment, the processor may shift the world coordinates, i.e., the three-dimensional coordinates of the blood vessel centerline 714, to coordinates having the rotational center of the imaging apparatus as a reference through Equation 6 below.

Xᵢₛₒ=D(X_{img},Cₚₒₛ)*SOD+Cₚₒₛ [Equation 6]

Here, Xᵢₛₒ may denote the coordinates having the rotational center of the imaging apparatus as a reference; X_{img} may denote the three-dimensional coordinates of the blood vessel centerline 714; Cₚₒₛ may denote the position information of the X-ray generator; SOD may denote the distance information between the X-ray generator and the subject; and D may denote the direction vector from the X-ray generator to the coordinates indicating the subject.

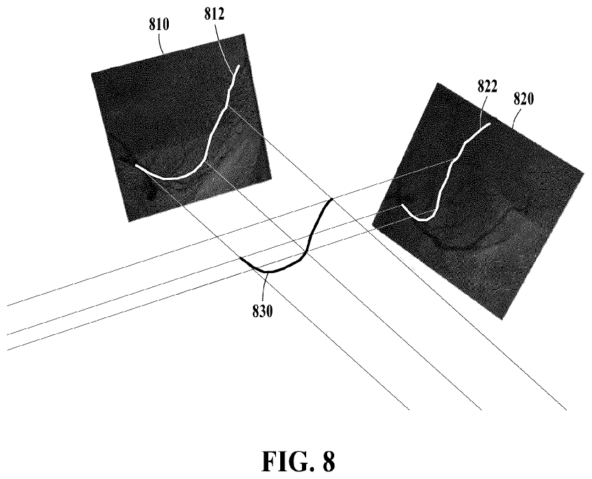

FIG. 8 is a diagram for explaining a method of obtaining height information in the gravity direction of a blood vessel from a plurality of blood vessel images according to an embodiment. Referring to FIG. 8, a processor (e.g., the processor 220 of FIGS. 2 and 3) of an electronic device (e.g., the electronic device 100 of FIGS. 1 and 2) for correcting a pressure difference value of a blood vessel may obtain height information in the gravity direction of a blood vessel included in a plurality of blood vessel images 810 and 820, based on imaging information (e.g., the imaging information 120 of FIG. 1) of an imaging apparatus (e.g., the imaging apparatus 400 of FIG. 4) for each of the plurality of blood vessel images 810 and 820 (e.g., the blood vessel image 110 of FIG. 1).

The processor may obtain calibration information and imaging direction information based on the imaging information. The processor may then detect a centerline 812 and a centerline 822 of a blood vessel included in each of the plurality of blood vessel images 810 and 820. The processor may then reconstruct the centerline 812 and the centerline 822 of the blood vessel included in each of the plurality of blood vessel images 810 and 820 into a three-dimensional centerline 830, based on the calibration information and the imaging information. For example, the processor may identify, based on a first centerline 812 of the blood vessel detected in a first image 810 captured from a first direction of the imaging apparatus among the plurality of blood vessel images 810 and 820, an epipolar line in a second image 820 captured from a second direction of the imaging apparatus among the plurality of blood vessel images 810 and 820. In addition, the processor may identify a common point of the first centerline 812 and the second centerline 822, based on the second centerline 822 of the blood vessel detected in the second image 820 and the epipolar line. Here, the epipolar line (or epiline) is a line along which an epipolar plane intersects with an image plane (e.g., the first image 810 and the second image 820), and when a point is given in one image plane (e.g., the first image 810), a corresponding point of that point must be located on the epipolar line of another image plane (e.g., the second image 820). That is, the processor may extract at least one feature point (or common point) by using geometric three-dimensional information through an epipolar plane passing through a first position of the imaging apparatus associated with capturing the first image 810, a second position of the imaging apparatus associated with capturing the second image 820, and a point on the blood vessel. In addition, the processor may generate the three-dimensional centerline 830 through triangulation based on the common point. The processor may then obtain height information in the gravity direction of the blood vessel based on the coordinates of the three-dimensional centerline 830.

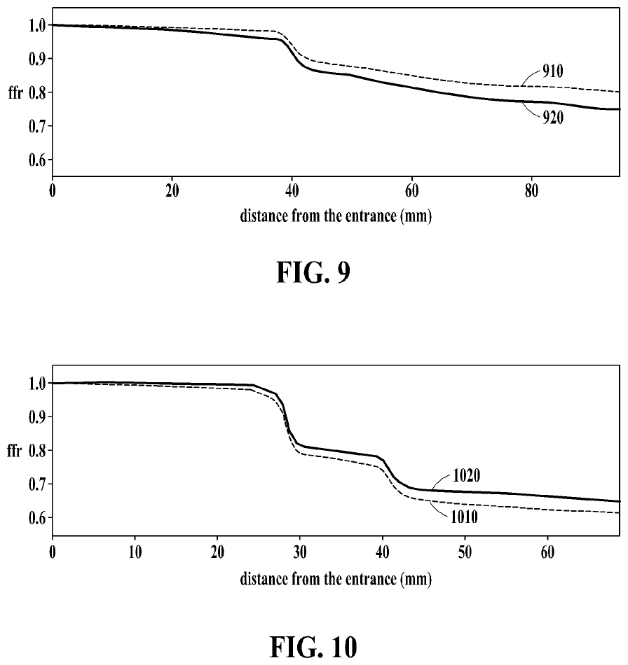

FIG. 9 is a diagram for explaining FFR in a blood vessel in which a pressure sensor is positioned higher than a blood vessel inlet according to an embodiment, and FIG. 10 is a diagram for explaining FFR in a blood vessel in which a pressure sensor is positioned lower than a blood vessel inlet according to an embodiment. Referring to FIGS. 9 and 10, blood flow feature values such as FFR may be used in the diagnosis of blood vessel lesions, such as coronary artery stenosis, for rapid and accurate diagnosis. FFR may be calculated by measuring the pressure difference between two points of a blood vessel (e.g., a proximal portion and a distal portion). In this case, in addition to the pressure loss due to the narrowing of the inner diameter of a lesion, a difference in pressure (e.g., hydrostatic pressure) depending on the vertical position of the blood vessel may affect the measurement results. For example, when measuring FFR, a pressure difference value due to gravity according to a difference in position of the pressure sensor, that is, a difference in height of the blood vessel, may affect the pressure difference value of the blood vessel. Accordingly, by correcting the pressure difference value of the blood vessel based on the pressure difference value due to gravity according to the difference in height of the blood vessel, more accurate FFR measurement may be supported.

FIG. 9 illustrates FFR in a blood vessel in which the position of the pressure sensor is located higher than the blood vessel inlet, for example, in the LAD. 910 in FIG. 9 may indicate FFR using the pressure difference value of the blood vessel excluding the pressure difference value due to gravity according to the difference in height of the blood vessel. In addition, 920 in FIG. 9 may indicate FFR using the pressure difference value of the blood vessel corrected based on the pressure difference value due to gravity according to the difference in height of the blood vessel. In the LAD, the position of the pressure sensor is located higher than the blood vessel inlet, so even if there is no pressure loss in the blood flow, the measurement may indicate that a pressure loss has occurred due to a hydrostatic pressure difference. Accordingly, a problem of overestimating FFR may occur in the LAD.

FIG. 10 illustrates FFR in a blood vessel in which the position of the pressure sensor is located lower than the blood vessel inlet, for example, in the LCx (or the RCA). 1010 in FIG. 10 may indicate FFR using the pressure difference value of the blood vessel excluding the pressure difference value due to gravity according to the difference in height of the blood vessel. In addition, 1020 in FIG. 10 may indicate FFR using the pressure difference value of the blood vessel corrected based on the pressure difference value due to gravity according to the difference in height of the blood vessel. In the LCx (or RCA), the position of the pressure sensor is located lower than the blood vessel inlet, and the LCx (or RCA) may have a tendency opposite to that of the LAD. For example, in the LCx (or RCA), FFR may be measured as larger than the actual value due to a hydrostatic pressure difference. Accordingly, in the LCx (or RCA), a problem of underestimating FFR may occur.

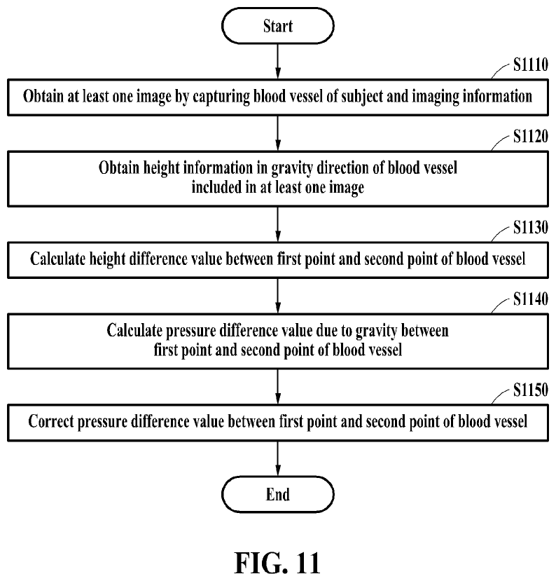

FIG. 11 is a diagram for explaining a method of correcting a pressure difference value of a blood vessel according to an embodiment. Referring to FIG. 11, a processor (e.g., the processor 220 of FIGS. 2 and 3) of an electronic device (e.g., the electronic device 100 of FIGS. 1 and 2) for correcting a pressure difference value of a blood vessel may, in step 1110 (S1110), obtain at least one image (e.g., the blood vessel image 110 of FIG. 1) by capturing the blood vessel of the subject and imaging information (e.g., the imaging information 120 of FIG. 1). For example, the processor may obtain an image of the blood vessel of the subject captured using an imaging apparatus (e.g., the imaging apparatus 400 of FIG. 4) while a contrast agent is injected into the blood vessel of the subject. In addition, the processor may obtain imaging information of the imaging apparatus at the time of capturing at least one image. Here, the imaging information may include, for example, at least one of distance information between an X-ray generator (e.g., the X-ray generator 454 of FIGS. 4 through 6) included in the imaging apparatus and an image acquisition apparatus (e.g., the image acquisition apparatus 452 of FIGS. 4 through 6) included in the imaging apparatus, distance information between the X-ray generator and the subject, position information of an examination table (e.g., the examination table 510 of FIGS. 5 and 6), pixel spacing information of the image acquisition apparatus, or angle information of the image acquisition apparatus with respect to the subject (e.g., the first rotation angle 522 and the second rotation angle 524).

In step 1120 (S1120), the processor may obtain height information in the gravity direction of the blood vessel included in at least one image. For example, the processor may, based on the imaging information, obtain height information in the gravity direction of the blood vessel included in the at least one image.

According to an embodiment, the processor may obtain calibration information and imaging direction information based on the imaging information of the imaging apparatus for the at least one image. The processor may then detect a centerline of the blood vessel included in the at least one image. The processor may then convert the centerline into three-dimensional coordinates based on the calibration information. For example, the processor may convert image coordinates in which the centerline is represented into camera coordinates, using an intrinsic parameter included in the calibration information. In this case, the intrinsic parameter may include a matrix generated based on distance information between the X-ray generator and the image acquisition apparatus and pixel spacing information of the image acquisition apparatus. In addition, the processor may convert the camera coordinates to world coordinates using an extrinsic parameter included in the calibration information. In this case, the extrinsic parameter may include a rotation matrix generated based on angle information of the image acquisition apparatus with respect to the subject and a transition vector indicating a camera position determined based on the rotation matrix and distance information between the X-ray generator and the subject. In addition, the processor may move the world coordinates to coordinates having the rotational center of the imaging apparatus as a reference, using the distance information between the X-ray generator and the subject and the position information of the X-ray generator. The processor may then obtain height information in the gravity direction of the blood vessel based on the three-dimensional coordinates of the centerline.

According to an embodiment, the processor may obtain calibration information and imaging direction information based on the imaging information of the imaging apparatus for each of the plurality of blood vessel images. The processor may then detect a centerline of the blood vessel included in each of the plurality of blood vessel images. The processor may then reconstruct the centerline of the blood vessel included in each of the plurality of blood vessel images into a three-dimensional centerline, based on the calibration information and the imaging information. For example, the processor may identify, based on a first centerline of the blood vessel detected in a first image captured from a first direction of the imaging apparatus among the plurality of blood vessel images, an epipolar line in a second image captured from a second direction of the imaging apparatus among the plurality of blood vessel images. In addition, the processor may, based on the second centerline of the blood vessel detected in the second image and the epipolar line, identify a common point of the first centerline and the second centerline. In addition, the processor may generate the three-dimensional centerline through triangulation based on the common point. The processor may then obtain height information in the gravity direction of the blood vessel based on the coordinates of the three-dimensional centerline.

In step 1130 (S1130), the processor may calculate a height difference value between the first point and the second point of the blood vessel. For example, the processor may calculate a height difference value between the first point and the second point of the blood vessel based on the obtained height information in the gravity direction of the blood vessel.

In step 1140 (S1140), the processor may calculate a pressure difference value due to gravity between the first point and the second point of the blood vessel. For example, the processor may calculate a pressure difference value due to gravity between the first point and the second point of the blood vessel by multiplying the height difference value between the first point and the second point of the blood vessel by the blood density of the blood vessel and the gravitational acceleration.

In step 1150 (S1150), the processor may correct the pressure difference value between the first point and the second point of the blood vessel. For example, the processor may calculate the pressure difference value between the first point and the second point of the blood vessel by adding the pressure difference value due to gravity to the pressure difference value due to the narrowing of the inner diameter of the lesion. Here, the pressure difference value due to the narrowing of the inner diameter of the lesion may be a value measured using FFR equipment. For example, the processor may calculate FFR based on the corrected pressure difference value of the blood vessel.

FIG. 12 is a diagram illustrating an artificial neural network model according to an embodiment. Referring to FIG. 12, an artificial neural network model 1200, as an example of a machine learning model, may represent a statistical learning algorithm implemented based on the structure of a biological neural network in machine learning technology and cognitive science, or a structure that executes the algorithm.

According to an embodiment, the artificial neural network model 1200 may represent a machine learning model having problem-solving capability, in which nodes that are artificial neurons forming a network through synaptic connections, similar to a biological neural network, repeatedly adjust synaptic weights and learn such that the error between a correct output and an inferred output corresponding to a given input is reduced. For example, the artificial neural network model 1200 may include any probabilistic model or neural network model used in artificial intelligence learning methods such as machine learning and deep learning.

According to an embodiment, the above-described model for correcting the pressure difference value of the blood vessel (or the model for calculating the pressure difference value due to gravity of the blood vessel) may be generated in the form of the artificial neural network model 1200. For example, the artificial neural network model 1200 may receive a blood vessel image obtained by capturing a blood vessel and imaging information of an imaging apparatus for the blood vessel image, estimate a pressure difference value due to gravity between a first point and a second point of the blood vessel based thereon, and correct the pressure difference value between the first point and the second point of the blood vessel based on the pressure difference value due to gravity.

The artificial neural network model 1200 may be implemented as a multi-layer perceptron (MLP) consisting of multiple layers of nodes and connections between the nodes. The artificial neural network model 1200, according to the embodiment, may be implemented using one of the artificial neural network model structures, including an MLP. The artificial neural network model 1200 may include an input layer 1220 that receives input data 1210 (or an input signal) from outside, an output layer 1240 that outputs output data 1250 (or an output signal) corresponding to the input data 1210, and n hidden layers 1230_1 to 1230_n (where n is a positive integer) positioned between the input layer 1220 and the output layer 1240, which receive signals from the input layer 1220, extract features, and transmit the features to the output layer 1240. Here, the output layer 1240 may receive signals from the n hidden layers 1230_1 to 1230_n and output the signals to the outside.

Training methods for the artificial neural network model 1200 may include a supervised learning method, in which the model is trained to optimize problem solving through input of correct teacher signals (or labels), and an unsupervised learning method, which does not require teacher signals. According to an embodiment, an electronic device according to an embodiment (e.g., the electronic device 100 of FIGS. 1 and 2) may train the artificial neural network model 1200 using a blood vessel image and imaging information of an imaging apparatus for the blood vessel image.

According to an embodiment, the electronic device may generate training data for training the artificial neural network model 1200. For example, the electronic device may generate a training data set including a blood vessel image and imaging information of an imaging apparatus for the blood vessel image. The electronic device may then train the artificial neural network model 1200 for calculating a pressure difference value due to gravity between the first point and the second point of the blood vessel based on the generated training data set. In addition, the electronic device may train the artificial neural network model 1200 for correcting a pressure difference value between the first point and the second point of the blood vessel based on the pressure difference value due to gravity.

According to an embodiment, the input variables of the artificial neural network model 1200 may include a blood vessel image in which a blood vessel is captured and imaging information of the imaging apparatus for the blood vessel image. When the input variables described above are input through the input layer 1220, the output variables output from the output layer 1240 of the artificial neural network model 1200 may be the pressure difference value due to gravity between the first point and the second point of the blood vessel, or the pressure difference value between the first point and the second point of the blood vessel corrected based on the pressure difference value due to gravity.

In this way, the plurality of input variables and the corresponding plurality of output variables are matched to the input layer 1220 and the output layer 1240 of the artificial neural network model 1200, respectively, and the synaptic values among the nodes included in the input layer 1220, the n hidden layers 1230_1 to 1230_n, and the output layer 1240 are adjusted, so that the artificial neural network model 1200 may be trained to extract a correct output corresponding to a given input. Through this training process, hidden characteristics in the input variables of the artificial neural network model 1200 may be identified. In addition, the synaptic values (or weights) among the nodes of the artificial neural network model 1200 may be adjusted so that the error between the output variables calculated based on the input variables and the target output is reduced. In addition, the electronic device may train an algorithm that receives as input a blood vessel image in which a blood vessel is captured and imaging information of the imaging apparatus for the blood vessel image, and may train the algorithm in a manner that minimizes the loss with respect to the pressure difference value due to gravity between the first point and the second point of the blood vessel, or the pressure difference value between the first point and the second point of the blood vessel corrected based on the pressure difference value due to gravity (i.e., annotation information). Using the artificial neural network model 1200 trained in this manner, the pressure difference value due to gravity between the first point and the second point of the blood vessel, or the pressure difference value between the first point and the second point of the blood vessel corrected based on the pressure difference value due to gravity, may be estimated.

The flowchart and description set forth above are merely illustrative examples, and some embodiments may be implemented differently. For example, in some embodiments, the order of each step may be changed, some steps may be performed repeatedly, some steps may be omitted, or some steps may be added.

The method described above may be provided as a computer program stored in a computer-readable storage medium for execution on a computer. The medium may continuously store a computer-executable program, or may temporarily store the program for execution or download. In addition, the medium may be any of various recording or storage means in the form of a single piece of hardware or a combination of several pieces of hardware, and is not limited to a medium directly connected to a computer system but may be distributed over a network. Examples of the medium include magnetic media such as hard disks, floppy disks, and magnetic tapes, optical recording media such as CD-ROMs and DVDs, magneto-optical media such as floptical disks, and media configured to store program instructions, including ROM, RAM, and flash memory. In addition, other examples of the medium include recording media or storage media managed by app stores that distribute applications, sites that supply or distribute various other types of software, and servers.

The methods, operations, or techniques of the disclosure may be implemented by various means. For example, such techniques may be implemented in hardware, firmware, software, or a combination thereof. Those of ordinary skill in the art will understand that the various illustrative logical blocks, modules, circuits, and algorithm steps described in connection with the disclosure may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends on the design requirements imposed on a given application and the overall system. Those of ordinary skill in the art may implement the described functionality in varying ways for each particular application, but such implementations should not be interpreted as causing a departure from the scope of the disclosure.

In a hardware implementation, the processing units used to perform the techniques may be implemented within one or more ASICs, DSPs, digital signal processing devices (DSPDs), PLDs, FPGAs, processors, controllers, microcontrollers, microprocessors, electronic devices, other electronic units designed to perform the functions described in the disclosure, a computer, or a combination thereof.

Accordingly, the various illustrative logic blocks, modules, and circuits described in connection with the disclosure may be implemented or performed with a general-purpose processor, a DSP, an ASIC, an FPGA or other PLDs, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

In firmware and/or software implementations, the techniques may be implemented as instructions stored on a computer-readable medium such as RAM, ROM, NVRAM, PROM, EPROM, EEPROM, flash memory, a CD, a magnetic or optical data storage device, or the like. The instructions may be executable by one or more processors and may cause the processor(s) to perform certain aspects of the functionality described in the disclosure.

If implemented in software, the techniques described above may be stored on or transmitted over a computer-readable medium as one or more instructions or code. Computer-readable media include both computer storage media and communication media, including any medium that facilitates transfer of a computer program from one place to another. Storage media may be any available media that can be accessed by a computer. By way of non-limiting examples, such computer-readable media may include RAM, ROM, EEPROM, CD-ROM or other optical disk storage, magnetic disk storage or other magnetic storage devices, or any other medium that can be used to carry or store desired program code in the form of instructions or data structures and that can be accessed by a computer. In addition, any connection is properly termed a computer-readable medium.

For example, if software is transmitted from a website, server, or another remote source using coaxial cable, fiber optic cable, twisted pair, digital subscriber line (DSL), or wireless technologies such as infrared, radio, and microwave, then coaxial cable, fiber optic cable, twisted pair, DSL, or wireless technologies such as infrared, radio, and microwave are included within the definition of medium. As used herein, disk and disc include CD, laser disc, optical disc, DVD, floppy disk, and Blu-ray disc, where disks usually reproduce data magnetically, while discs reproduce data optically with lasers. Combinations of the above should also be included within the scope of computer-readable media.

A software module may reside in RAM memory, flash memory, ROM memory, EPROM memory, EEPROM memory, registers, a hard disk, a removable disk, a CD-ROM, or any other form of storage medium known in the art. An illustrative storage medium may be coupled to a processor such that the processor can read information from, or write information to, the storage medium. Alternatively, the storage medium may be integrated into the processor. The processor and the storage medium may be in an ASIC. The ASIC may be in a user terminal. Alternatively, the processor and the storage medium may be discrete components in a user terminal.

Although the embodiments described above have been described as utilizing aspects of the presently disclosed subject matter in one or more standalone computer systems, the disclosure is not limited thereto and may be implemented in conjunction with any computing environment, such as a network or distributed computing environment. Furthermore, aspects of the subject matter in the disclosure may be implemented in a plurality of processing chips or devices, and storage may similarly be affected across a plurality of devices. Such devices may include personal computers (PCs), network servers, and portable devices.

Although the disclosure has been described in connection with some embodiments herein, various modifications and changes may be made without departing from the scope of the disclosure as understood by a person skilled in the art to which the disclosure pertains. In addition, such modifications and changes should be considered to fall within the scope of the claims appended hereto.

What is claimed is:

1. A method of correcting a pressure difference value of a blood vessel, performed by at least one processor, the method comprising:
obtaining at least one image of a blood vessel of a subject captured by using an imaging apparatus, and imaging information of the imaging apparatus regarding the at least one image;
obtaining, based on the imaging information, height information in a gravity direction of the blood vessel included in the at least one image;
calculating, based on the height information, a height difference value between a first point and a second point of the blood vessel;
calculating, based on the height difference value, a pressure difference value due to gravity between the first point and the second point of the blood vessel; and
correcting, based on the pressure difference value due to gravity, a pressure difference value between the first point and the second point of the blood vessel to obtain a corrected pressure difference value.

2. The method of claim 1, wherein the imaging information comprises at least one of first distance information between an X-ray generator included in the imaging apparatus and an image acquisition apparatus included in the imaging apparatus, second distance information between the X-ray generator and the subject, position information of an examination table, pixel spacing information of the image acquisition apparatus, or angle information of the image acquisition apparatus with respect to the subject.

3. The method of claim 1, wherein the imaging information comprises a first distance information between an X-ray generator included in the imaging apparatus and an image acquisition apparatus included in the imaging apparatus, a second distance information between the X-ray generator and the subject, a pixel spacing information of the image acquisition apparatus, and an angle information of the image acquisition apparatus with respect to the subject.

4. The method of claim 3, wherein the obtaining of the height information comprises:
obtaining, based on the imaging information, calibration information and imaging direction information;
detecting a centerline of the blood vessel included in the at least one image;
converting, based on the calibration information, the centerline into three-dimensional coordinates; and
obtaining, based on the three-dimensional coordinates of the centerline, the height information of the blood vessel in the gravity direction.

5. The method of claim 4, wherein the converting of the centerline into the three-dimensional coordinates comprises:
converting, by using an intrinsic parameter included in the calibration information, image coordinates in which the centerline is represented into camera coordinates, wherein the intrinsic parameter comprises a matrix generated based on the first distance information and the pixel spacing information;
converting, by using an extrinsic parameter included in the calibration information, the camera coordinates into world coordinates, wherein the extrinsic parameter comprises a rotation matrix generated based on the angle information, and a transition vector indicating a camera position determined based on the rotation matrix and the second distance information; and
moving, by using the second distance information and position information of the X-ray generator, the world coordinates to coordinates having a rotational center of the imaging apparatus as a reference.

6. The method of claim 1, wherein the at least one image comprises a plurality of images obtained by capturing the blood vessel of the subject from different directions using the imaging apparatus, and
the obtaining of the height information comprises:
obtaining, based on imaging information of the imaging apparatus for each of the plurality of images, calibration information and imaging direction information;
detecting a centerline of the blood vessel included in each of the plurality of images;
reconstructing, based on the calibration information and the imaging direction information, the centerline of the blood vessel included in each of the plurality of images into a three-dimensional centerline; and
obtaining, based on coordinates of the three-dimensional centerline, the height information in the gravity direction of the blood vessel.

7. The method of claim 6, wherein the reconstructing of the centerline of the blood vessel included in each of the plurality of images into the three-dimensional centerline comprises:
identifying, based on a first centerline of the blood vessel detected in a first image captured from a first direction of the imaging apparatus among the plurality of images, an epipolar line in a second image captured from a second direction of the imaging apparatus among the plurality of images;
identifying, based on a second centerline of the blood vessel detected in the second image and the epipolar line, a common point of the first centerline and the second centerline; and
generating the three-dimensional centerline through triangulation based on the common point.

8. The method of claim 1, wherein the calculating of the pressure difference value due to gravity comprises calculating the pressure difference value due to gravity between the first point and the second point of the blood vessel by multiplying the height difference value by a density of blood and a gravitational acceleration.

9. The method of claim 1, further comprising calculating information related to a blood flow characteristic based on the corrected pressure difference value.

10. A non-transitory computer-readable storage medium storing instructions that, when executed by a computer, cause the computer to perfume the method of claim 1.

11. An electronic device comprising:
at least one processor; and
memory storing instructions, wherein the instructions, when executed by the at least one processor, cause the electronic device to:
obtain at least one image of a blood vessel of a subject captured by using an imaging apparatus, and imaging information of the imaging apparatus regarding the at least one image;
obtain, based on the imaging information, height information in a gravity direction of the blood vessel included in the at least one image;
calculate, based on the height information, a height difference value between a first point and a second point of the blood vessel;
calculate, based on the height difference value, a pressure difference value due to gravity between the first point and the second point of the blood vessel; and
correct, based on the pressure difference value due to gravity, a pressure difference value between the first point and the second point of the blood vessel to obtain a corrected pressure difference value.

12. The electronic device of claim 11, wherein the imaging information comprises at least one of first distance information between an X-ray generator included in the imaging apparatus and an image acquisition apparatus included in the imaging apparatus, second distance information between the X-ray generator and the subject, position information of an examination table, pixel spacing information of the image acquisition apparatus, or angle information of the image acquisition apparatus with respect to the subject.

13. The electronic device of claim 11, wherein the imaging information comprises a first distance information between an X-ray generator included in the imaging apparatus and an image acquisition apparatus included in the imaging apparatus, a second distance information between the X-ray generator and the subject, a pixel spacing information of the image acquisition apparatus, and an angle information of the image acquisition apparatus with respect to the subject.

14. The electronic device of claim 13, wherein the instructions, when executed by the at least one processor, further cause the electronic device to:
obtain, based on the imaging information, calibration information and imaging direction information;
detect a centerline of the blood vessel included in the at least one image;
convert, based on the calibration information, the centerline into three-dimensional coordinates; and
obtain, based on the three-dimensional coordinates of the centerline, the height information of the blood vessel in the gravity direction.

15. The electronic device of claim 13, wherein the instructions, when executed by the at least one processor, further cause the electronic device to:
convert, by using an intrinsic parameter included in the calibration information, image coordinates in which the centerline is represented into camera coordinates; convert, by using an extrinsic parameter included in the calibration information, the camera coordinates into world coordinates; and
move, by using the second distance information and position information of the X-ray generator, the world coordinates to coordinates having a rotational center of the imaging apparatus as a reference, wherein the intrinsic parameter comprises a matrix generated based on the first distance information and the pixel spacing information, and the extrinsic parameter comprises a rotation matrix generated based on the angle information, and a transition vector indicating a camera position determined based on the rotation matrix and the second distance information.

16. The electronic device of claim 11, wherein the at least one image comprises a plurality of images obtained by capturing the blood vessel of the subject from different directions using the imaging apparatus, and
the instructions, when executed by the at least one processor, further cause the electronic device to:
obtain, based on imaging information of the imaging apparatus for each of the plurality of images, calibration information and imaging direction information;
detect a centerline of the blood vessel included in each of the plurality of images; reconstruct, based on the calibration information and the imaging direction information, the centerline of the blood vessel included in each of the plurality of images into a three-dimensional centerline; and
obtain, based on coordinates of the three-dimensional centerline, the height information in the gravity direction of the blood vessel.

17. The electronic device of claim 16, wherein the instructions, when executed by the at least one processor, cause the electronic device to:
identify, based on a first centerline of the blood vessel detected in a first image captured from a first direction of the imaging apparatus among the plurality of images, an epipolar line in a second image captured from a second direction of the imaging apparatus among the plurality of images;
identify, based on a second centerline of the blood vessel detected in the second image and the epipolar line, a common point of the first centerline and the second centerline; and
generate the three-dimensional centerline through triangulation based on the common point.

18. The electronic device of claim 11, wherein the instructions, when executed by the at least one processor, cause the electronic device to calculate the pressure difference value due to gravity between the first point and the second point of the blood vessel by multiplying the height difference value by a density of blood and a gravitational acceleration.

19. The electronic device of claim 11, wherein the instructions, when executed by the at least one processor, cause the electronic device to calculate information related to a blood flow characteristic based on the corrected pressure difference value.
